(19) United States
(12) Patent Application Publication
(10) Pub. No.: US 2016/0202225 A1
(43) Pub. Date: Jul. 14, 2016

(54) System for Detecting a Gas and Method Therefor

(71) Applicant: Case Western Reserve University, Cleveland, OH (US)

(72) Inventors: Philip X.L. Feng, Beachwood, OH (US); Zenghui Wang, Shaker Heights, OH (US); Jaesung Lee, Cleveland Heights, OH (US)

(21) Appl. No.: 14/993,064

(22) Filed: Jan. 11, 2016

Related U.S. Application Data
(60) Provisional application No. 62/101,642, filed on Jan. 9, 2015.

(51) Int. Cl. G01N 33/00; G01N 27/407; G01N 21/31; G01N 27/416

(52) U.S. Cl. CPC G01N 33/0032; G01N 27/4166; G01N 27/4072; G01N 21/31; G01N 2201/06113

(57) ABSTRACT

An apparatus and method for detecting a gas with high sensitivity, high SNR, and low cost is disclosed. Embodiments of the present invention include sensor nodes that communicate with a common controller, where each sensor node includes a resonant sensor that comprises a resonator having a selectively chemisorptive layer disposed upon it. The chemisorptive layer is a nanoparticle-based layer that improves the trapping probability for target-gas molecules, thereby improving the correspondence of the resonance frequency of the resonator to the gas concentration in the atmosphere in which it resides, and improving the sensitivity of the resonant sensor as compared to prior-art resonant mass sensors. Measurement of an electrical parameter of the chemisorption layer can also be used as a secondary detection mode. By employing the chemisorptive layer as an efficient and selective mass-collection layer affords embodiments of the present invention improved noise immunity.

### CROSS REFERENCE TO RELATED APPLICATIONS

This application claims the benefit of U.S. Provisional Application Ser. No. 62/101,642, filed Jan. 9, 2015, entitled “Real-Time Monitoring of Methane Leak by Ultrasensitive SiC MEMS Gas Analyzer Networks with Wireless Communication” (Attorney Docket 747-010PR1), which is incorporated herein by reference. If there are any contradictions or inconsistencies in language between this application and one or more of the cases that have been incorporated by reference that might affect the interpretation of the claims in this case, the claims in this case should be interpreted to be consistent with the language in this case.

### FIELD OF THE INVENTION

The present invention relates to sensors in general, and, more particularly, to MEMS gas sensors.

### BACKGROUND OF THE INVENTION

The ability to detect the presence of a gas with high fidelity and high sensitivity is critical in many applications, such as mining, refining, petroleum transport, and homeland defense. An ability to detect methane gas, for example, is considered crucial for industrial and residential gas safety, and greenhouse gas emission control. Methane (CH₄) is estimated to be the second largest contributor to global warming and it is estimated that methane accounted for 8.8% of the global warming impact from domestic human activity in 2011.

Many different gas detection devices and systems have been developed and such systems commonly obtain a sample of the ambient atmosphere and then analyze the sample to determine the presence or absence of the particular gas in question. Conventional methods of analysis include optical absorption spectral analysis, infrared and mid-infrared techniques, optical frequency combs, various mass spectrometers, resonant mass detection, and electrochemical detection.

Spectral-analysis-based detection is based on the fact that every material absorbs light at wavelengths that are characteristic of that material. By measuring the absorption of specific wavelength components by a material, therefore, the molecular composition of material can be identified and quantified with great accuracy. Unfortunately, while such systems are capable of high sensitivity, conventional spectral-analysis systems are complicated, bulky, and generally quite expensive.

Gas chromatography and mass spectrometry have typically been considered as the gold standards for gas analysis which is done by measuring mass/charge ratio. These approaches are capable of high resolution; however the instruments are usually highly expensive and bulky, and require special, complicated preparation and treatment of samples such as electron spray ionization and multiple stages of vacuum.

Resonant mass detection using micro/nanoscale resonators is based on the fact that a resonant element will resonate (i.e., vibrate) at a frequency that is determined by its mass and stiffness. Shorter (less massive) guitar strings, for example, resonate at higher frequencies than longer guitar strings. As a result, by tracking changes in the resonant frequency of a resonant element as gas molecules adsorb onto its surface, the concentration of gas near the element can be determined. Resonant mass detection systems in the prior art have demonstrated sensitivities as low as zeptogram (10⁻²¹ grams) levels in controlled environments, such as ultra-high vacuum and ultra-low temperature. Unfortunately, resonators known in the prior art are generally based on one-dimensional nanostructures (e.g., thin nanometer-scale-width wires, and molecular-scale nanotubes) characterized by very low sticking and trapping probabilities for gas molecules on their surfaces. In addition, typical prior-art resonant mass sensors have extremely small surface areas on which the gas can adsorb. As a result, improvement in the sensitivity of conventional resonant mass sensors will be challenging.

Electrochemical gas sensors are based on material layers whose electrical properties change in response to adsorption of a particular gas. Prior-art methane detectors, for example, monitor the resistance of a layer of metal oxide, such as tin oxide, gallium oxide, beryllium oxide, and the like, which changes as a function of adsorbed gas. Unfortunately, such electrochemical gas detection often suffers from limited sensitivity (in the range of 100 parts-per-million (ppm)—i.e., a few percent of the Lower Explosive Limit (LEL) for methane, for example), and is easily perturbed by local magnetic and electric fields. As a result, such sensors are unable to detect at the single ppm level or parts-per-billion (ppb) levels, which is critical in many applications, such as for the early detection of explosive-gas leakage in open spaces.

A low-cost, lightweight gas sensor having high fidelity, high sensitivity, and long lifetime would be an important advance in the state of the art of gas detection.

### SUMMARY OF THE INVENTION

The present invention enables detection of a target gas without some of the costs and disadvantages of gas sensors known in the prior art. Embodiments of the present invention have extremely high sensitivity, can operate at high speed, are low power, and can be small and light. Gas sensors in accordance with the present invention are particularly well suited for use in applications such as petroleum well monitoring (i.e., down-hole monitoring), well pad monitoring, distributed pollution monitoring, breath analyzers, and the like. Further, sensor nodes in accordance with the present invention can be very small and light, thereby making them suitable for deployment on manned and/or unmanned vehicles (e.g., airplanes, trucks, cars, all-terrain vehicles, unmanned ground vehicles (UGVs), unmanned aerial vehicles (UAVs), autonomous robots, etc.).

Gas sensors in accordance with the present invention comprise at least one resonator that includes a chemisorptive layer that is substantially selective for a particular gas of interest (i.e., a target gas). The combination of a resonator and chemisorptive layer affords embodiments of the present invention significant advantages over prior-art gas sensors. First, compared to conventional resonator-based gas sensors, the addition of a chemisorptive layer provides selectivity to the target gas. Second, in contrast to chemisorption-based gas sensors of the prior art, the principal mode of gas detection is based on a change of mass rather than an electrochemical change of the layer in response to adsorption of the target gas. Since electrochemical detection can be affected by many other factors (e.g., temperature, pressure, humidity, stray electric fields, electro-static discharge, etc.), the present invention enables more sensitive and noise-immune detection of the target gas. Third the use of a chemisorptive layer that includes a plurality of nanoparticles increases the effective surface area on which the target gas can be adsorbed, thereby increasing the responsivity and sensitivity of the resonant sensor. Fourth, the fact that the chemisorptive layer also undergoes an electrochemical change in the presence of the target gas enables the use of electrochemical detection as a secondary mode for determining the concentration of the gas, thereby improving noise immunity.

An illustrative embodiment of the present invention is a methane sensor system that includes one or more sensor nodes and a controller. The sensor nodes and controller communicate wirelessly, which enables the sensor nodes to be easily distributed around a detection region, or mounted on a movable platform, such as a vehicle, autonomous robot, UAV, and the like. Each sensor node includes a sensor comprising a silicon-carbide resonator and a first layer disposed on the resonator, where the first layer is selectively chemisorptive for methane. In the illustrative embodiment, the first layer includes tin oxide nanoparticles, which gives the first layer a high surface-area-to-volume ratio that facilitates adsorption of gas molecules. The sensor node also includes drive electronics for driving the resonator into resonance, readout electronics for tracking the resonance of the resonator, and a transceiver for wirelessly communicating with the controller.

In some embodiments, a plurality of sensor nodes are included, wherein at least one sensor node is selective for gas other than methane. In some embodiments, at least one sensor node includes multiple gas sensors that are collectively sensitive for at least two different gasses.

In some embodiments, the chemisorptive layer is doped with a catalyst to improve sensitivity and enhance the layers rejection of gasses other than the target gas.

In some embodiments, at least one sensor node is in communications with the controller via a wired connection.

In some embodiments, multi-modal detection of a gas is enabled by including electrodes for electrically interrogating the chemisorptive layer. The electrodes enable measurement of an electrical parameter of the first layer to provide a secondary mode of gas detection.

In some embodiments, the first layer and a mirror are arranged to collectively define an optically resonant cavity through which gas flow is enabled. The optically resonant cavity is interrogated by a multi-spectral light signal that gives rise to a second light signal whose spectral content is based on the spectral absorption characteristics of the gasses included in the gas flow. The second light signal is analyzed by a spectrometer and the controller to determine a chemical characteristic of the gas flow. In some embodiments, the mirror is a surface of an optical fiber with which the sensor is operatively coupled.

An embodiment of the present invention is an apparatus comprising a first sensor that includes: a first resonator; and a first layer disposed on the first resonator, the first layer comprising a plurality of nanoparticles that collectively enable selective chemisorption of a first gas; wherein a first resonance frequency of the first resonator is based on the mass of the first layer; and wherein the first sensor is operative for providing a first signal that is indicative of a first concentration of the first gas at a first location.

Another embodiment of the present invention is an apparatus comprising: a first sensor node that includes: a first gas sensor having a first resonance frequency that is based on the selective chemisorption of a first gas by a first layer; a first electronic module operative for determining the first resonance frequency; and a first transceiver for providing a first output signal to a controller, the first output signal being based on the first resonance frequency; and the controller, wherein the controller is operative for generating an estimate of a first concentration of the first gas at a first location based on the first output signal.

Yet another embodiment of the present invention is a method comprising: providing a first layer disposed on a first resonator, the first layer comprising a plurality of nanoparticles that collectively enable selective chemisorption of a first gas; determining a first resonance frequency of the first resonator, the first resonance frequency being based on the mass of the first layer at a first location; and estimating a first concentration of the first gas at the first location based on the first resonance frequency.

### BRIEF DESCRIPTION OF THE DRAWINGS

FIGS. 1A-B depict schematic drawings of a resonant-mass sensor, with and without surface functionalization, respectively.

FIGS. 1C-D depict photographs of a portion of a resonator in accordance with the present invention, before and after, respectively, formation of adsorption layer 108.

FIG. 1E schematically depicts the improved responsivity of resonator 100 to the presence of gas 106 when using a functionalized surface.

FIG. 1F depicts a plot of nanoparticle size versus anneal temperature.

FIG. 2 depicts a schematic diagram of a multimodal gas sensor system in accordance with the present invention.

FIGS. 3A-B depict schematic drawings of a top and side view, respectively, of sensor 208.

FIG. 4A depicts a schematic drawing of a circuit suitable for detecting and tracking the resonance frequency of a resonator in accordance with the illustrative embodiment of the present invention.

FIG. 4B depicts a schematic drawing of an alternative electrical drive/readout circuit suitable for use with the present invention.

FIG. 4C depicts a schematic drawing of another alternative electrical drive/readout circuit suitable for use with the present invention.

FIG. 4D depicts a schematic drawing of yet another alternative electrical drive/readout circuit suitable for use with the present invention.

FIG. 5 depicts operations of a method suitable for monitoring a gas in a detection region in accordance with the illustrative embodiment of the present invention.

FIG. 6A depicts a schematic drawing of a distributed sensor system in accordance with another alternative embodiment of the present invention.

FIG. 6B depicts a detailed view of sensor 606.

FIG. 6C depicts an illustration of an exemplary sensor system deployment in accordance with the alternative embodiment of the present invention.

FIG. 7 depicts a schematic drawing of a circuit suitable for optically detecting and tracking the resonance frequency of a resonator in accordance with the illustrative embodiment of the present invention.

FIG. 8 depicts operations of a method in accordance with optically interrogated embodiments of the present invention.

FIG. 9A depicts a schematic drawing of an enlarged view of the arrangement of sensor 606 in accordance with a first optical method for monitoring the motion of resonator 608.

FIG. 9B depicts a schematic drawing of an enlarged view of the arrangement of sensor 606 in accordance with a second optical method for monitoring the motion of resonator 608.

FIG. 10 depicts methods of an operation suitable for detecting a gas via a spectroscopic detection mode.

### DETAILED DESCRIPTION

### Operating Principal of the Invention

The fundamental operating principle of the present invention is that functionalizing the adsorption surface of a resonant mass sensor with the addition of a selectively chemisorptive layer enables gas detection with significantly improved sensitivity and selectivity as compared to gas sensors of the prior art.

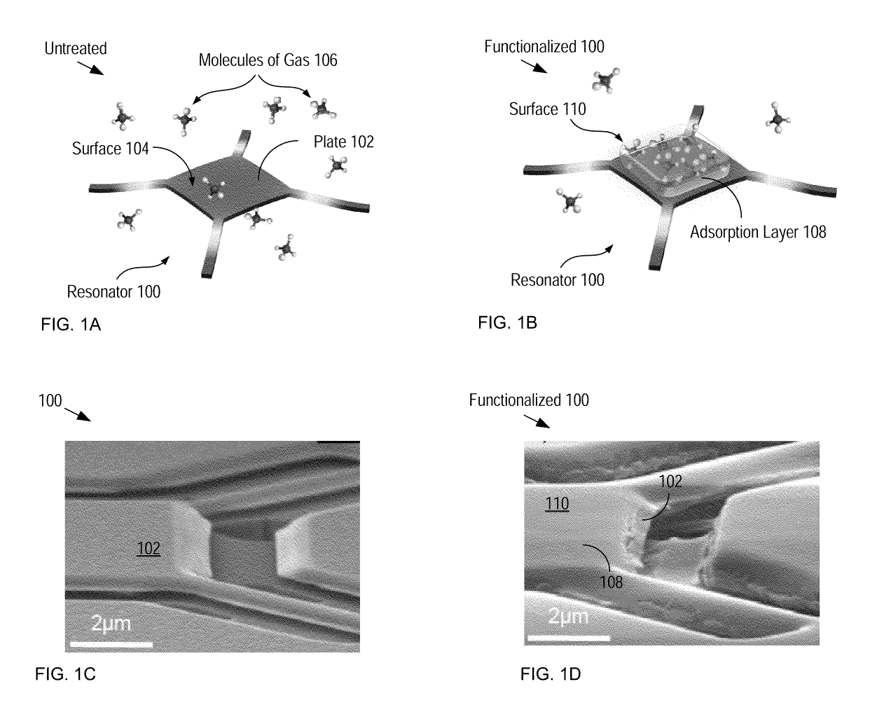

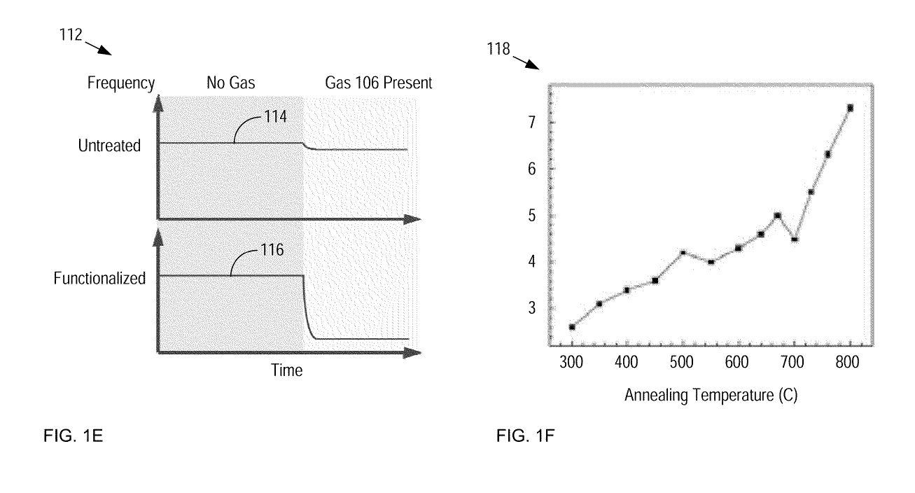

FIGS. 1A-B depict schematic drawings of a resonant-mass sensor, with and without surface functionalization, respectively. Resonator 100 is a trampoline-type resonant-mass gas sensor having central plate 102, which includes adsorption surface 104. The resonance frequency of sensor 100 is based, primarily on the mass of plate 102. As a result, as molecules of gas 106 adsorb on surface 104, the mass of the plate increases and the resonance frequency of resonator 100 decreases. As depicted in FIG. 1A, untreated surface 104 is characterized by very low sticking and trapping probabilities for molecules of target gas 106. As a result, the sensitivity of the untreated resonator is relatively poor.

FIGS. 1C-D depict photographs of a portion of a resonator in accordance with the present invention, before and after, respectively, formation of adsorption layer 108.

It is an aspect of the present invention that the addition of adsorption layer 108 to surface 102 gives rise to a functionalized surface (i.e., surface 110) having improved sticking and trapping probabilities, however. As depicted in FIG. 1B, surface 110 is highly effective for adsorbing molecules of gas 106, thereby enabling gas detection with improved sensitivity and selectivity as compared to gas sensors of the prior art.

FIG. 1E depicts the improved responsivity of resonator 100 to the presence of gas 106 when using a functionalized surface. Plot 112 includes traces 114 and 116, which depict the response of resonator 100 to the introduction of gas 106 with an untreated surface and functionalized surface, respectively. As indicated by plot 112, the addition of adsorption layer 108 to surface 104 enables significantly improved responsivity to the target gas.

It is another aspect of the present invention that using a nanoparticle-based chemisorption layer that selectively adsorbs a target gas (i.e., gas 106) to functionalize surface 104 provides additional advantages over prior-art gas sensors. Specifically, a layer of nanoparticles is characterized by a greater total surface area by virtue of the nanopores included in the layer. As a result, the use of a nanoparticle-based adsorption layer significantly increases the surface area available for adsorption of gas molecules. For example, an adsorption layer that includes nanoparticles having diameters in the range of 3 to 14 nm gives rise to an average surface area as high as 194 m²/g. As a result, 2.7 grams of such nanoparticles has a total surface area of approximately 531.1 m²—approximately equal to the area of a standard football field, including its end zones. It should be noted that the diameter of the nanoparticles can be controlled by adjusting the annealing temperature used during their formation.

FIG. 1F depicts a plot of nanoparticle size versus anneal temperature. Plot 118 shows the grain size of the nanoparticles within adsorption layer 108, as measured using x-ray spectrometer and based on the (101) peak. Plot 118 evinces that grain size can be controlled over the range of approximately 3 nm to approximately 7.4 nm by controlling anneal temperature over a range from 300° C. to 800° C.

Further, as discussed in more detail below, the addition of a selective chemisorption layer to the surface of a microresonator enables multi-modal detection capability to the device—specifically, an improved resonant mass detection mode, as well as a substantially independent electrochemical detection mode.

### Remote Distributed Gas Sensing

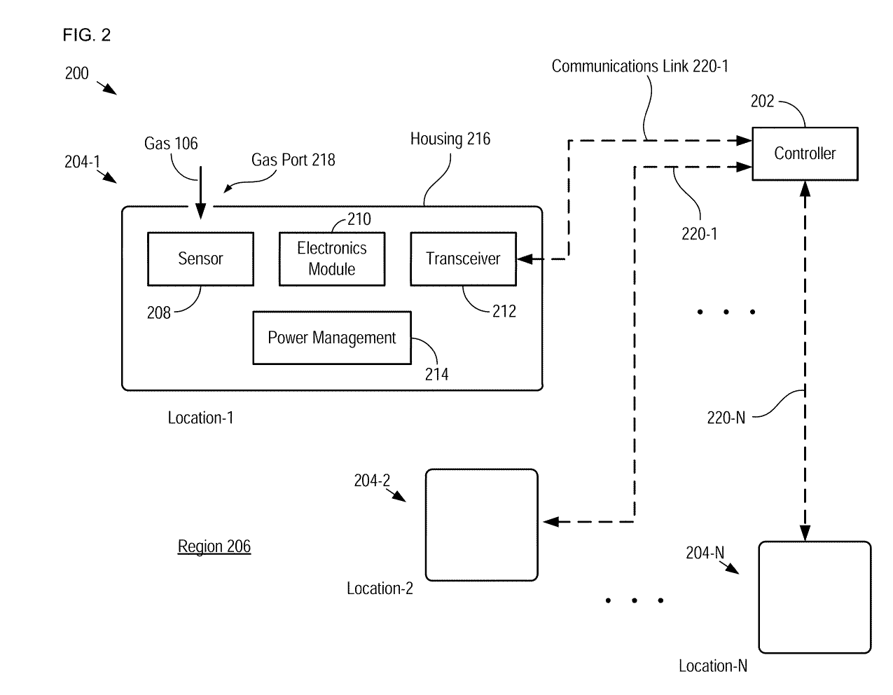

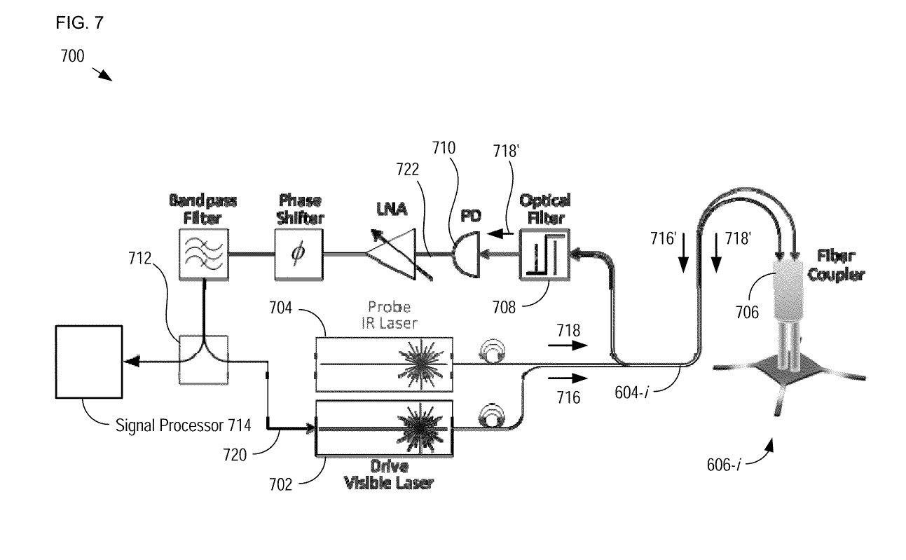

FIG. 2 depicts a schematic diagram of a multimodal gas sensor system in accordance with the present invention. System 200 is operative for remotely detecting the presence and concentration of a target gas throughout a detection region using two substantially independent detection modes—resonant mass detection and electrochemical detection. System 200 includes controller 202 and sensor nodes 204-1 through 204-N, where N is the number of sensor nodes 204 distributed throughout detection region 206. Detection region 206 might be, for example, a petroleum processing plant, off-shore oil rig, a manufacturing plant, and the like. System 200 is an example of a sensor system in accordance with the present invention that is based on a resonant mass sensor that is both driven and readout electronically. In should be noted, however, that a resonant mass sensor in accordance with the present invention can alternatively be driven and readout optically, which affords additional advantages to such embodiments, as discussed below and with respect to FIG. 7.

Controller 202 is a conventional processing system operative for receiving output signals from one or more of sensors nodes 204-1 through 204-N (referred to, collectively, as sensor nodes 204), processing the output signals, and transmitting the processed information to an external server (not shown), such as a cloud server, central computing system, etc. In some embodiments, controller 202 is operative for performing analysis of the output signals and/or developing a temporally correlated spatial map of gas concentration within region 206. Such a spatial map can be used to determine, for example, detection of gas leaks within region 206, where the gas leaks are occurring, their severity, etc.

Each of sensor nodes 204 is a multi-modal gas detection module that is selectively sensitive to gas 106 such that it can monitor the concentration of the gas via multiple detection modes. As depicted in exemplary sensor node 204-1, each sensor node includes sensor 208, electronics module 210, transceiver 212, and power management system 214, all of which are contained in housing 216. Sensor 208 is fluidically coupled to the ambient environment at its location via gas port 218 (e.g., sensor node 204-1 is fluidically coupled to the ambient environment at location 1, etc.). In the depicted example, gas 106 is methane; however, it will be clear to one skilled in the art, after reading this Specification, how to specify, make, and use alternative embodiments of the present invention that are sensitive to other gasses. Further, it will be clear how to specify, make, and use alternative embodiments wherein sensor node 204 includes multiple sensors 208, at least one of which is selectively sensitive to a different gas than at least one other sensor. In some embodiments, multiple sensors 208, each of which is sensitive to the same gas, are included in sensor node 204 to enable redundancy and/or signal averaging.

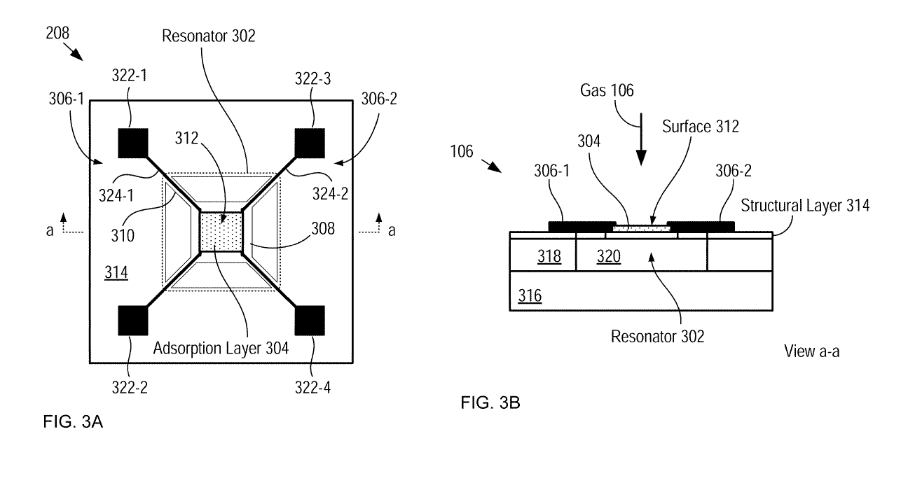

Sensor 208 is analogous to sensor 100, described above; however, sensor 208 includes electrodes that enable it to be electrically driven into resonance, have its resonance frequency electrically readout, and have its resistance measured to provide a secondary gas detection mode. Sensor 208 is described in more detail below and with respect to FIGS. 3A-B.

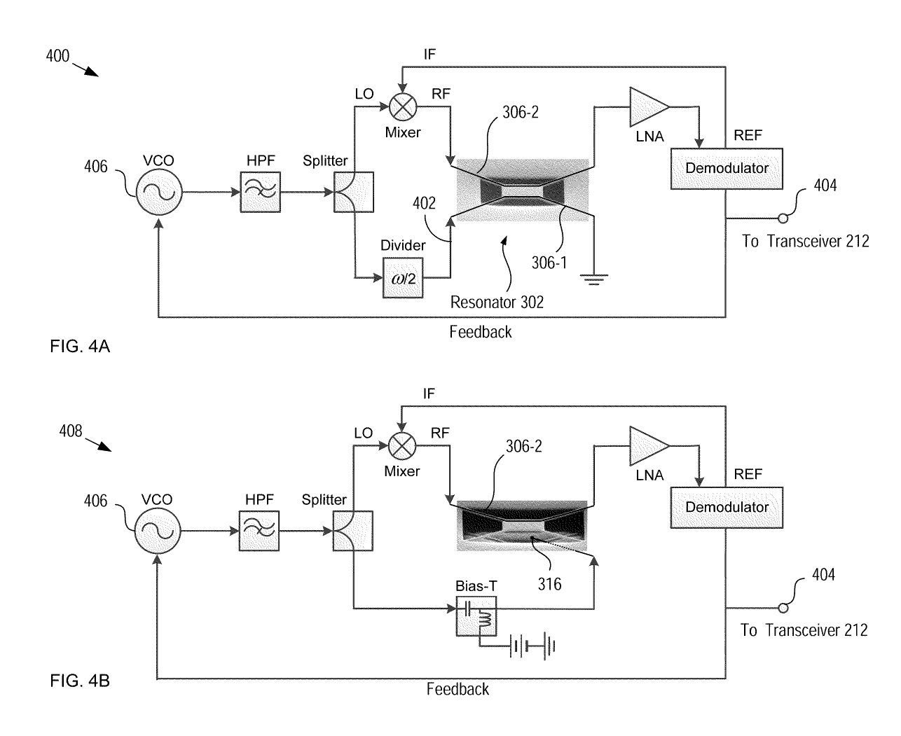

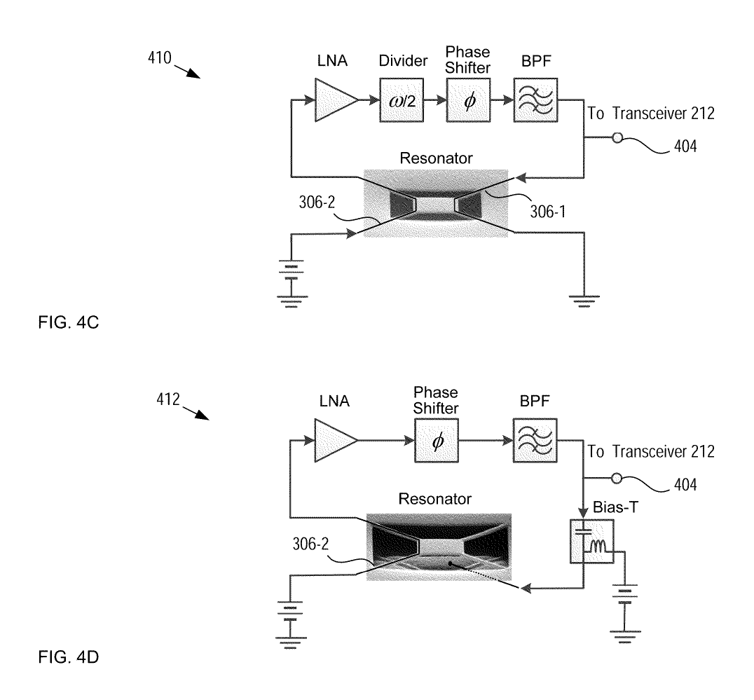

Electronics module 210 is an application-specific integrated circuit (ASIC) that includes drive and readout electronics for enabling real-time sensing of the mass of the resonator of sensor 208. In the depicted example, electronics module 210 includes circuitry for driving the resonator of sensor 208 into resonance, as well as detector electronics for detecting an adsorption-induced shift in the resonance frequency of the resonator and measuring the resistance of adsorption layer 108. In the depicted example, electronics module 210 monitors the resonance frequency of the resonator using a feedback loop that provides a self-adjusting drive signal. In other words, sensor 208 and electronics module 210 collectively define a closed-loop oscillator, which enables operation with high frequency stability and high resolution. Examples of electronics modules suitable for use in embodiments of the present invention are described by Yang, et al., in “Zeptogram-Scale Nanomechanical Mass Sensing,” Nano Letters, Vol. 6, No. 4, pp. 583-586 (2006), which is incorporated herein by reference. Electronics module 210 is described in more detail below and with respect to FIGS. 4A-D.

Transceiver 212 is a wireless transceiver operative for communicating with controller 202 via communications link 220. Transceiver 212 provides controller 202 an output signal indicative of the concentration of gas 106 at the location of sensor node 204.

Power management system 214 is a module suitable for powering electronics module 108 and transceiver 212, as well as providing other necessary control functions. In the depicted example, power management system 214 includes a battery for providing power to sensor node 204; however, it will be clear to one skilled in the art, after reading this Specification, how to specify, make, and use alternative embodiments wherein power management system 214 includes a different power source, such as a piezoresistive energy scavenging and storage system, an inductively coupled energy storage system, and the like.

Housing 216 is a conventional weatherproof enclosure that includes gas port 116, which fluidically couples sensor 208 with the ambient environment at the location of sensor node 204. Preferably, housing 216 is made from a strong, lightweight material that facilitates the mounting of sensor node 204 on a mobile platform, such as a UAV. It will be clear to one skilled in the art, after reading this Specification, how to specify, make, and use housing 216.

FIGS. 3A-B depict schematic drawings of a top and side view, respectively, of sensor 208. The side view shown in FIG. 3B is taken through line a-a, as depicted in FIG. 3A. Sensor 208 is a multi-modal sensor that includes resonator 302, adsorption layer 304, and electrodes 306-1 and 306-2. Sensor 208 is analogous to functionalized resonator 100; however, the inclusion of electrodes 306-1 and 306-2 enables the resonator to be electronically driven and readout. It also enables two substantially independent detection modes for monitoring gas 106—resonant mass detection and electrochemical detection. In some embodiments, sensor 208 monitors gas 106 using only resonant mass detection. In other words, sensor 208 is a “multi-modal sensor.” Multi-modal detection or sensing, as used herein, means the detection has multiple modalities, i.e., sensing mechanisms, embedded in the same device structure, and enabled by hybrid signal transduction schemes enabled on the same device. One sensing modality is based on monitoring the resonance frequency shift induced by the gas adsorption enhanced by a plurality of nanoparticles. The other sensing modality is to examine the electrochemical properties (voltage, impedance) of the layer of the nanoparticles.

Embodiments of the present invention include a detection mode that is based upon a resonant sensor that detects a change in mass of a resonator due to the adsorption of molecules of a gas of interest (i.e., gas 106) on its surface. Such resonant mass sensors are well known in the prior art and examples are described by, among others, Naik, et al., in “Towards single-molecule nanomechanical mass spectroscopy,” Nature Nanotechnology, Vol. 4, pp. 445-450 (2009), and Yang, et al., in “Zeptogram-Scale Nanomechanical Mass Sensing,” Nano Letters, Vol. 6, No. 4, pp. 583-586 (2006), which is incorporated herein by reference.

The sensitivity of prior-art resonant mass sensors is limited by the fact that the probability of trapping gas molecules on a non-functionalized surface is relatively low, however. In addition, prior-art devices are typically operated only in high vacuum and low temperature. Further, the signal-to-noise ratio (SNR) achievable with prior art resonant mass sensors is typically poor due to a lack of selectivity for the gas of interest. Still further, many prior-art resonant mass sensors have limited surface area available for the adsorption of target-gas molecules.

As discussed above, the present invention enables significantly improved sensitivity with high SNR by adding a layer that is selectively chemisorptive for gas 106 (i.e., adsorption layer 108) to resonator 302. As a result, the trapping probability for molecules of gas 106 is significantly improved. This improves sensitivity for gas 106 while simultaneously reducing noise that would result from the adsorption of unwanted gas molecules on the resonator surface. In addition, chemisorption layers in accordance with the present invention are preferably nanoparticle-based. As a result, they give rise to a dramatic increase in the effective surface area available for the adsorption of target gas as compared to resonant gas sensors of the prior art. In contrast to the prior art, additionally, sensors in accordance with the present invention can operate in ambient conditions.

Resonator 302 is trampoline-type resonant element comprising plate 308 and tethers 310, which are held above substrate 316 by sacrificial layer 318. Resonator 302 is a portion of structural layer 314 that has been defined using a conventional subtractive patterning technique (e.g., focused-ion beam (FIB) etching, deep-reactive-ion etching (DRIE), etc.). Once formed, resonator 302 is made mechanically active (i.e., movable with respect to substrate 316) by forming cavity 320 in sacrificial layer 318 using a sacrificial etch in accordance with conventional microfabrication techniques. In some embodiments, structural layer 314 is disposed directly on substrate 316 and cavity 320 is formed by removing a portion of the substrate under plate 308 and tethers 310 using a suitable substrate etch. In some embodiments, cavity 320 is formed by completely removing substrate 316 under the plate/tether region using crystallographic-dependent etching, DRIE, and the like. In some embodiments, the manner in which cavity 320 is formed is dependent upon operational considerations, such as desired quality factor, Q, of the resonator, ambient pressure, etc.

In the depicted example, structural layer 314 is a layer of silicon carbide (SiC) having a thickness of approximately 500 nm, plate 308 is substantially square with sides of approximately 8 microns, and tethers 310 are approximately 8 microns long and 1 micron wide. Sacrificial layer 318 has a thickness of approximately 500 nm, which determines the quiescent separation between plate 308 and substrate 316.

One skilled in the art will recognize that the materials, shape, dimensions, and characteristics of resonator 302 are matters of design choice and that myriad alternative choices are suitable for use for resonator 302 without departing from the scope of the present invention. For example, in some embodiments, structural layer 314 comprises a different material suitable for use in resonator 302 (e.g., silicon, quartz, compound semiconductors, metals, composite materials, ceramic materials, etc.). In some embodiments, resonator 302 has a shape other than a trampoline, such as a substantially continuous membrane, a doubly-supported beam, a cantilever, and the like. Further, in some embodiments, plate 308 has a shape other than a square, such as a circle, oval, rectangle, ellipse, etc.

One skilled in the art will recognize, after reading this Specification, that the sensitivity of sensor 208 scales with the total surface area available for adsorption of gas molecules (for a given total initial mass). Further, sensitivity scales with the ratio of the mass of a molecule of target gas to the total initial mass of resonator 302. Preferably, therefore, resonator 302 has a large surface area available for adsorption of gas molecules, while the thickness of structural layer 314 is kept as thin as possible to minimize its total initial mass. As a result, in some embodiments, plate 308 is encompasses a relatively large percentage of the area of resonator 302.

Adsorption layer 304 is a layer of material that is substantially selectively chemisorptive for gas 106. Adsorption layer 304 includes a plurality of nanoparticles that enable the selective chemisorption of gas 106. In the depicted example, adsorption layer 304 is disposed on resonator 302 as a layer of tin oxide nanoparticles having a thickness of approximately 50 nm. Preferably, adsorption layer 304 has a thickness within the range of a few nm to a few μm, although other thicknesses are within the scope of the present invention. In some embodiments, sensor 106 is selectively sensitive to a gas other than methane. In such embodiments, adsorption layer comprises a different material that is suitably selective for the desired target gas.

As discussed above, the use of a nanoparticle-based adsorption layer affords embodiments of the present invention with significant advantages over the prior art by increasing the total surface area available for adsorption of target-gas molecules. In the depicted example, adsorption layer 304 includes tin oxide nanoparticles having diameters of approximately 3 nm. On the surface of plate 308, therefore, a 2 μm-thick coating of 3 nm-diameter nanoparticles yields a total area of approximately 1 mm². This is an increase in the surface area of plate 308 by approximately 10⁴×, which significantly enhances the capture rate of gas 106. Further, the O_{2ad}-, O_{ad}- or O_{ad2}-ions (at high temperature) readily available on tin oxide surface due to oxygen absorption react actively with methane, which gives rise to efficient chemisorption of the methane gas, resulting in high detection sensitivity.

It should be noted that the use of metal-oxide films for the detection of chemical gasses is well known in the prior art. For example, the use of tin oxide layers to detect methane is described in numerous publications, including U.S. Pat. No. 4,535,315, and by C. Wang, et al., in “Metal Oxide Gas Sensors: Sensitivity and Influencing Factors,” Sensors, Vol. 10, pp., 2088-2106 (2010), each of which is incorporated herein by reference. In such conventional gas sensors, detection of the target gas is achieved by monitoring an electrical parameter of the metal-oxide layer, which can be affected by many factors other than the adsorption of the target gas. As a result, the sensitivity and accuracy of prior-art gas sensors is degraded by changes in temperature, humidity, local electrical fields, etc. In addition, over time, responsivity of such prior-art gas sensors degrades significantly as the metal-oxide layer becomes “poisoned” by the adsorption of target gas.

It is an aspect of the present invention, however, that detection of a target gas is based primarily on the change in mass of a resonator due to adsorption of gas molecules on its surface. The change in mass manifests as a change in the resonance frequency of resonator 302, which can be measured with high precision, and which is independent from electrochemical detection based on a change in an electrical parameter of adsorption layer 304. Measurement of such a change in an electrical parameter of the layer can be used to supplement resonant mass detection to provide additional confirmation and minimize false alarms. Embodiments of the present invention, therefore, can have better sensitivity and improved SNR as compared to prior-art metal oxide-based gas sensors. As a result, the present invention enables detection of gas concentrations that are much lower than possible in the prior art, making early detection of even small gas leaks a possibility. Further, although the trapping probability of adsorption layer 304 decreases as its surface becomes saturated with molecules of gas 106, sensor 208 can continue to detect the presence of target gas in the same manner as prior-art resonant gas sensors.

In the depicted example, adsorption layer 304 is formed on resonator 302 by spin coating a sol-gel containing tin oxide nanoparticles onto structural layer 314. The nascent adsorption layer is then dried and calcinated to yield the finished chemisorption layer. Typically, sol-gel synthesis is based on either the hydrolysis of tin alkoxide or, preferably, hydrolysis of tin (IV) chloride. Although the illustrative embodiment includes an adsorption layer comprising tin oxide nanoparticles, it will be clear to one skilled in the art, after reading this Specification, how to specify, make, and use alternative embodiments of the present invention wherein adsorption layer includes nanoparticles of a different material. Materials suitable for use in the present invention include, without limitation, tungsten oxide, zinc oxide, rare-earth oxides, and the like.

It is another aspect of the present invention that by choosing proper spin-coating and annealing conditions, an adsorption layer having desired thickness, nanoparticle size, and nanopore density can be attained. It will be clear to one skilled in the art, after reading this Specification, that the detection sensitivity and efficiency of sensor 208 are affected by the material properties of adsorption layer 304; therefore, the ability to control the parameters of the adsorption layer enables fabrication of sensors having a wide range of operating characteristics.

Although adsorption layer 304 is preferably formed using spin coating, in some embodiments, adsorption layer 304 is formed using a different deposition method. Deposition methods suitable for use with the present invention include, without limitation, atomic-layer epitaxy, vapor-phase epitaxy, sputter deposition, chemical-vapor deposition, plasma-enhanced chemical-vapor deposition, and the like.

It should be noted that, while the use of a nanoparticle-based adsorption layer is preferable, other selectively chemisorptive layers can be used without departing from the scope of the present invention.

In some embodiments, adsorption layer 304 is doped with a catalyst, such as cobalt, etc., to enhance the detection of gas 106, as well as mitigate noise due to adsorption of other gases (e.g., longer chain hydrocarbons, carbon monoxide (CO), etc.). In some embodiments, sensor 208 includes one or more additional resonators 302 whose adsorption layer is doped differently to make it selective for a different gas to effect a differential-selectivity capability. This enables sensor 208 to better discriminate gas 106 from potentially confounding gasses.

In the depicted example, electrodes 306 can be used as ohmic heaters to desorb gas molecules from adsorption layer 304, thereby performing a “reset” of a “poisoned” sensor. In some embodiments, sensor 208 includes a separate heater that is thermally coupled with plate 308 and adsorption layer 304. A heating capability also enables operating the sensor at an elevated temperature to further improve its selectivity to certain target gasses (e.g., methane).

Once adsorption layer 304 is fully formed on resonator 302, the adsorption layer can be optionally patterned using a conventional etching technique, such as focused-ion-beam (FIB) etching, wet etching, reactive-ion etching (RIE), and the like. In some embodiments, adsorption layer 304 is left unpatterned.

Electrodes 306-1 and 306-2 are disposed on resonator 302 such that they are operatively coupled with plate 308 and adsorption layer 304. Electrode 306-1 includes contact pads 322-1 and 322-2 and trace 324-1. Electrode 306-2 includes contact pads 322-3 and 322-4 and trace 324-2. Electrode 306-1 is operative for driving resonator 302 into resonance via thermal actuation, while electrode 306-2 is operative for piezoresistively monitoring the resonance frequency of resonator 302. Electrodes 306 are electrically coupled with electronics 108.

FIG. 4A depicts a schematic drawing of a circuit suitable for detecting and tracking the resonance frequency of a resonator in accordance with the illustrative embodiment of the present invention. Circuit 400 includes a portion of electronics module 108 and sensor 208, which are operatively coupled to collectively define a phase lock loop suitable for driving resonator 302 into resonance and tracking its resonance frequency. Circuit 400 is arranged to electrothermally actuate the resonator and detect its position piezoresistively.

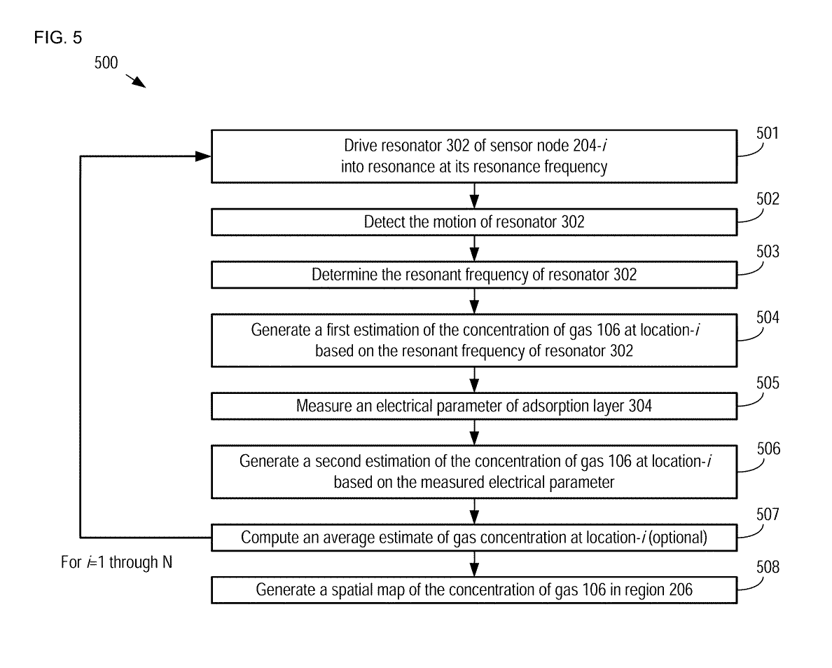

FIG. 5 depicts operations of a method suitable for monitoring a gas in a detection region in accordance with the illustrative embodiment of the present invention. Method 500 is described with continuing reference to FIGS. 2-4A. Method 500 begins with operation 501, wherein at each sensor node 204-i, where i=1 through N, resonator 302 is driven into resonance at its resonance frequency. It should be noted that, when referring to resonator 302 in the description of method 500, the resonator is meant to include adsorption layer 304, as well as any adsorbed molecules of gas 106, the masses of which affect the resonance frequency of resonator 302.

Circuit 400 drives resonator 302 into resonance by modulating its temperature via drive signal 402, which is applied to electrode 306-1.

At operation 502, the motion of resonator 302 is detected as a change in the resistance of electrode 306-2, which chances as a function of induced strain based on the piezoresistance effect.

At operation 503, circuit 400 provides output signal 404 to transceiver 212, where output signal 404 reflects the resonance frequency of resonator 302. As discussed above, the resonance frequency of resonator 302 is based on the mass of plate 308 and adsorption layer 304, as well as molecules of gas 106 adsorbed on surface 312. As a result, output signal 404 is representative of the amount of gas 106 adsorbed on adsorption layer 304.

As gas molecules adsorb on the resonator, circuit 400 detects the shift in the resonance frequency of the resonator, via the phase lock loop, and alters output signal 404 to reflect the frequency shift. Output signal 404 is also used to control VCO 406, which drives resonator 302. This negative feedback loop enables VCO 406 to track the frequency of resonator 302 as its resonance frequency shifts in response to adsorbed gas molecules.

At operation 504, transceiver 212 transmits output signal 404 to controller 202 on communications link 220-i. Controller 202 uses output signal 404 to deduce the amount of gas adsorbed to the resonator and uses this deduction to generate a first estimate of the concentration of gas 106 at location-i.

The components of the oscillator circuit are normally included in electronics module 210, in the form of a circuit board or even an application-specific integrated circuit (ASIC); however, in some embodiments, these components are included in sensor node 204 as a stand-alone module. In some embodiments some or all of the components of the oscillator circuit are included in controller 202.

At operation 505, an electrical parameter of adsorption layer 304 is measured in conventional fashion by circuit elements of circuit 400 (not shown). In the depicted example, this electrical parameter is the resistance between electrodes 306-1 and 306-2, the value of which is based on the amount of gas 106 adsorbed on the surface of adsorption layer 304. In some embodiments, an electrical parameter other than the resistance between electrodes 306 is measured in operation 505. The value of the resistance is then provided to transceiver 212, which transmits it to controller 202 on communications link 220-i.

At operation 506, a second estimate of the concentration of gas 106 at location-i is made based on the resistance measured between electrodes 306.

At optional operation 507, controller 202 computes an average estimate of gas concentration at location-i based on the first, second, and third estimates.

At operation 508, controller 202 generates a spatial map of the concentration of gas 106 in detection region 206 based on the average estimates of gas concentration at location-1 through location-N and the times at which the estimates were made. In some embodiments, at least one of sensor nodes 204 is mounted on a movable platform, such as a UAV, which enables the sensor node to provide a series of gas-concentration estimations to the controller for use in developing the spatial map of gas concentration.

In the depicted example, resonator 302 is driven into resonance electrothermally and its motion is detected piezoresistively. It will be clear to one skilled in the art, however, after reading this Specification, how to specify, make, and use alternative embodiments that employ different driving and/or detection mechanisms. Driving and/or detection mechanisms suitable for use in embodiments of the present invention include, without limitation, piezoelectric, electrostatic, capacitive mechanical, magnetomotive, electromagnetic, and magnetoresistive, among others.

Further, in the depicted example, device resonance is sustained and detected using phase lock loop. It will be clear to one skilled in the art, however, after reading this Specification, how to specify, make, and use alternative embodiments wherein the frequency tracking mechanism is based on a different scheme. Frequency-tracking mechanisms suitable for use in embodiments of the present invention include, without limitation, feedback oscillation, open-loop frequency sweep, pulse-driven (ring-down) time-domain measurement, and the like.

FIG. 4B depicts a schematic drawing of an alternative electrical drive/readout circuit suitable for use with the present invention. Circuit 408 is a phase lock loop circuit that is analogous to circuit 400; however, circuit 408 drives resonator 302 into resonance by applying a modulated electric field between electrode 306-2 and substrate 316. This voltage gives rise to an electrostatic force on resonator 302 that is suitable for electrically driving resonator 302 into resonance.

FIG. 4C depicts a schematic drawing of another alternative electrical drive/readout circuit suitable for use with the present invention. Circuit 410 includes a portion of electronics module 210 and sensor 208, which collectively define a closed-loop oscillator suitable for driving resonator 302 into resonance and outputs its frequency signal, like a clock circuit. As in circuit 400, circuit 410 actuates the motion of resonator 302 electrothermally and detects its motion piezoresistively. The closed-loop oscillator of circuit 410 feeds back the resonance signal and amplifies it to drive resonator 302, and provides an AC signal as output signal 404, which is at the same frequency as the resonance of the resonator.

FIG. 4D depicts a schematic drawing of yet another alternative electrical drive/readout circuit suitable for use with the present invention. Circuit 412, together with sensor 208, defines a closed-loop electrical oscillator circuit that is analogous to circuit 410; however, circuit 412 drives resonator 302 into resonance by applying a modulated electric field between electrode 306-2 and substrate 316 to electrically drive the resonator into resonance.

As mentioned briefly above, in addition to the electrically based drive and sense schemes herein, resonant mass sensors in accordance with the present invention can also be driven and readout optically, which affords additional advantages to such embodiments. The use of an optical readout, for example, enables a third substantially independent gas detection mode. This is attained by arranging the sensor in a gas-spectroscopy arrangement in which the light used to readout the motion of the resonator passes through a chamber that is fluidically coupled with the environment at the location of the sensor node. As a result, absorption of wavelengths characteristic of gas 106 can be detected. In addition, by using one side of resonator 302 as one mirror of an optically resonant cavity, the absorption signal is amplified by the optical resonance that occurs within the cavity. Further, by using only optical signals to interact with sensors 208, the sensor nodes are made substantially immune to electromagnetic interference.

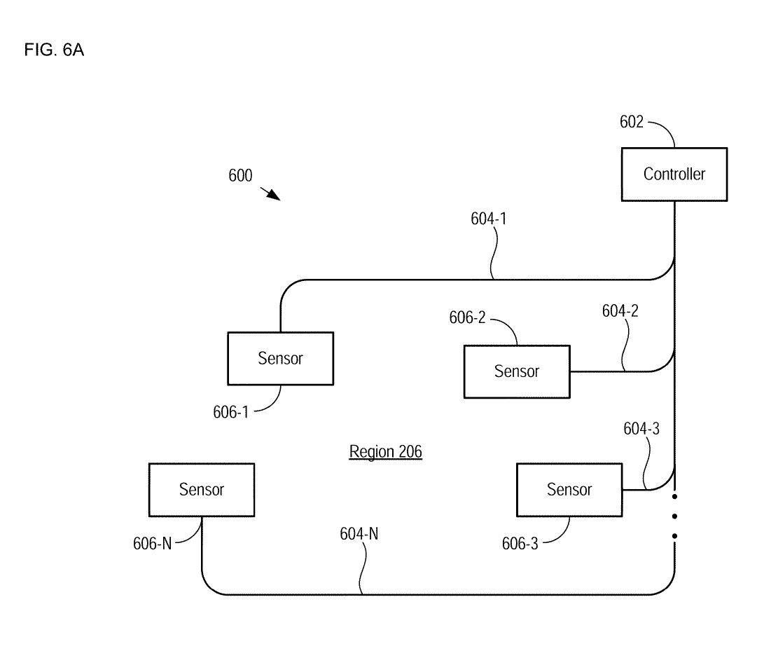

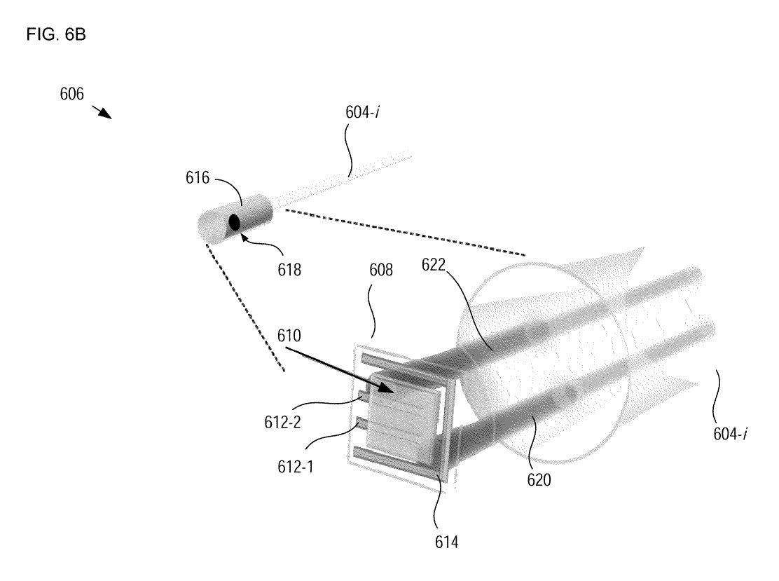

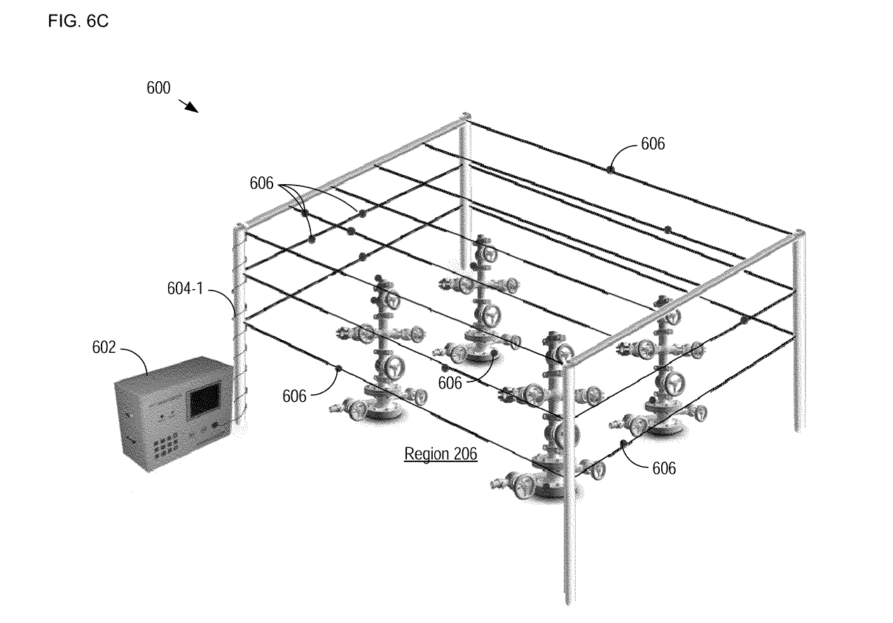

FIG. 6A depicts a schematic drawing of a distributed sensor system in accordance with another alternative embodiment of the present invention. Sensor system 600 includes controller 602, fibers 604-1 through 604-N, and sensors 606-1 through 606-N. Sensor system 600 enables monitoring of gas 106 in region 206 employing three modes of chemical detection—resonant-mass detection, electrochemical detection, and spectroscopic detection.

Controller 602 is analogous to controller 202, described above and with respect to FIG. 2; however, controller 602 includes all of the electrical and optical components necessary for the operation of sensors 606, such as a drive laser, a probe laser, laser modulators, frequency tracking circuitry, detector, a processor, and a transceiver. In some embodiments, controller 602 also includes an optical fiber switching element that enables it to interrogate each sensor 606 individually.

Controller 602 is optically coupled with each of sensors 606 via a different one of conventional multimode optical fibers 604-1 through 604-N (referred to, collectively, as optical fibers 604).

Optical fibers 604 fan out from the controller and each includes a sensor 606 disposed at its end facet. Controller 602 includes one drive laser and one probe laser, whose output light signals are shared among sensors 606; however, in some embodiments, controller 602 includes a different drive laser and probe laser (and associated closed-loop oscillator and signal processing system) for each of sensors 606.

FIG. 6B depicts a detailed view of sensor 606. Each of sensors 606 is analogous to sensor 208 described above; however, sensor 606 is configured for optical interrogation (i.e., optical drive and readout). Sensor 606 includes resonator 608, adsorption layer 610, electrodes 612-1 and 612-2, heater 614, and housing 616.

Resonator 608 is analogous to resonator 302 and adsorption layer 610 is analogous to adsorption layer 304 described above. In the depicted example, resonator 608 is a substantially complete membrane of structural material on which an adsorption layer 610 is disposed.

Electrodes 612-1 and 612-2 are electrodes formed such that they are operative for measuring the resistance of the adsorption layer between them. As a result, electrodes 612-1 and 612-2 enable electrochemical detection of gas 106.

Heater 614 is a conventional ohmic heater element disposed on resonator 608. Heater 614 enables the device to be heated to induce desorption of adsorbates when desired.

Although not shown in FIG. 6B, each of electrodes 612 and heater 614 is electrically connected to controller 602 by a conductive trace routed to sensor 606 along with optical fiber 604.

Housing 616 is a small, weatherproof housing comprising gas inlet 618. Housing 616 contains the components of sensor 606 such that resonator 608 is located at the free end of its associated fiber and able to receive each of optical drive signal 620 and optical sense signal 622 from optical fiber 604.

Optical fibers 604 can be easily mounted to infrastructure at the detection region to deploy sensors 606 in virtually any desired arrangement. In similar fashion, optical fibers 604 and their associated sensors 606 can trail from an aircraft or UAV while the vehicle flies above detection region 206.

FIG. 6C depicts an illustration of an exemplary sensor system deployment in accordance with the alternative embodiment of the present invention. The optical fiber connected sensor network make it easy to deploy in field by simply positioning the fibers in any applicable surfaces, without the requirement of optical alignment, such as is required in prior-art systems based on free space lasers.

FIG. 7 depicts a schematic drawing of a circuit suitable for optically detecting and tracking the resonance frequency of a resonator in accordance with the illustrative embodiment of the present invention. Oscillator circuit 700 and sensor 606 collectively define a closed-loop oscillator suitable for driving resonator 608 into resonance and tracking its resonance frequency. Circuit 700 includes drive laser 702, probe laser 704, fiber coupler 706, wavelength filter 708, detector 710, splitter 712, and signal processor 714. Circuit 700 optionally includes various additional circuit elements for signal conditioning purposes, such as a low-noise amplifier, phase shifter, and bandpass filter, as indicated. The components of the oscillator circuit are included in the controller, and are sequentially connected to each sensor node of choice using a switch.

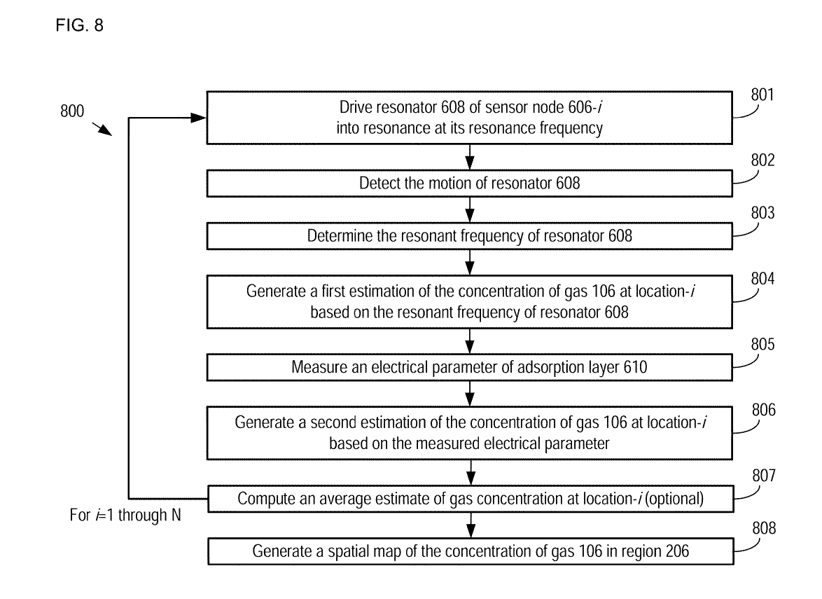

FIG. 8 depicts operations of a method in accordance with optically interrogated embodiments of the present invention. Method 800 begins with operation 801, wherein resonator 608 is driven into resonance with optical drive signal 716, which is provided by drive layer 702.

To drive resonator 608 into resonance, the intensity of optical drive signal 716 is modulated via drive signal 720 to excite resonant motion of resonator 608 at its resonance frequency by virtue of induced heating of the resonator surface. In the depicted example, light signal 716 has a wavelength of approximately 405 nm.

At operation 802, the motion of resonator 608 is detected.

To monitor the motion of resonator 608, probe laser 704 provides light signal 718 to sensor 606-i via optical fiber 604-i. In the depicted example, light signal 718 has a wavelength of approximately 633 nm.

One skilled in the art will recognize that the wavelengths of light signals 716 and 718 are merely exemplary and that other wavelengths can be used without departing from the scope of the present invention. Further, in the illustrative embodiment, lasers 716 and 718 are optically coupled with sensor 606-i using optical fiber; thus, sensor 606-i is fiber coupled directly to controller 602, which contains the required electronics to generate an estimate of gas concentration at each of location-1 through location-N, and use a switch to select and operate each sensor nodes.

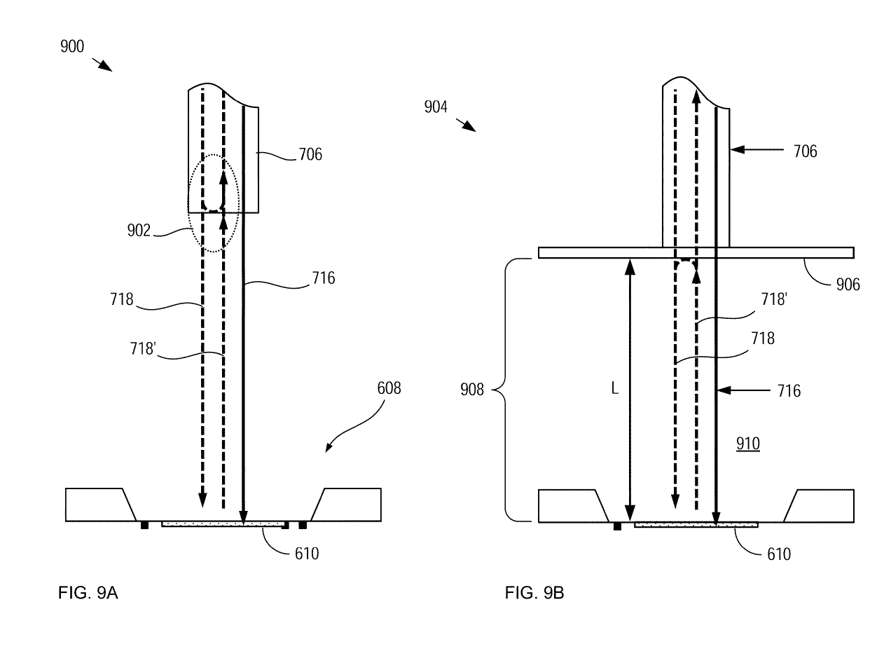

FIG. 9A depicts a schematic drawing of an enlarged view of the arrangement of sensor 606 in accordance with a first optical method for monitoring the motion of resonator 608. Arrangement 900 shows an arrangement of sensor 606 and fiber coupler 706 that enables detection of the motion of resonator 608 using an interferometry method.

Light signals 716 and 718 are provided to the back surface of resonator 608 by conventional fiber coupler 706. As light signal 718′ couples back into fiber coupler 706, interference of light signals 718 and 718′ occurs in region 902, giving rise to an intensity modulation of light signal 718′ that is based on the instantaneous position of resonator 608 and, therefore, its motion.

Light signal 716′ and 718′ are conveyed to optical filter 708 where light signal 716′ is substantially blocked. As a result, photodetector 710 receives only light signal 718′.

Photodetector 710 detects light signal 718′ and provides corresponding electrical signal 722. The output of detector 710 is amplified and conditioned and provided to signal processor 714 as electrical signal 720, which is also used as the drive signal for drive laser 702, thereby providing closed-loop feedback that enables the drive laser to track changes in the resonance frequency of resonator 608 due to adsorption of gas molecules.

At operation 803, signal processor 714 determines the resonance frequency of resonator 608 based on the frequency of electrical signal 720.

At operation 804, controller generates a first estimate of gas concentration at location-i based on the resonance frequency determined in operation 803.

At operation 805, the resistance between electrodes 612-1 and 612-2 is measured.

At operation 806, a second estimate of gas concentration at location-i based on the resistance measured between electrodes 612-1 and 612-2.

At optional operation 807, an average estimate of gas concentration at location-i is computed from the first and second estimates.

At operation 808, a spatial map of the concentration of gas 106 is developed for region 206, as described above.

FIG. 9B depicts a schematic drawing of an enlarged view of the arrangement of sensor 606 in accordance with a second optical method for monitoring the motion of resonator 608. Arrangement 904 depicts an arrangement of sensor 606 and fiber coupler 706 that collectively defines an optically resonant cavity that enables measurement of the absorption characteristics of gas 106 when it is located within the cavity.

In arrangement 904, fiber coupler 706 terminates at mirror 906. In some embodiments, the mirror is the end facet of the fiber coupler.

Resonator 608 and mirror 906 are arranged in close proximity such that they collectively define a low-Q optically resonant cavity 908 whose cavity length, L, is determined by the position of resonator 608 and adsorption layer 610. In some embodiments, a high-Q optically resonant cavity is defined by resonator 608 and mirror 906. For example, a bulk mirror can be used to form an optically resonant cavity with sensor 606.

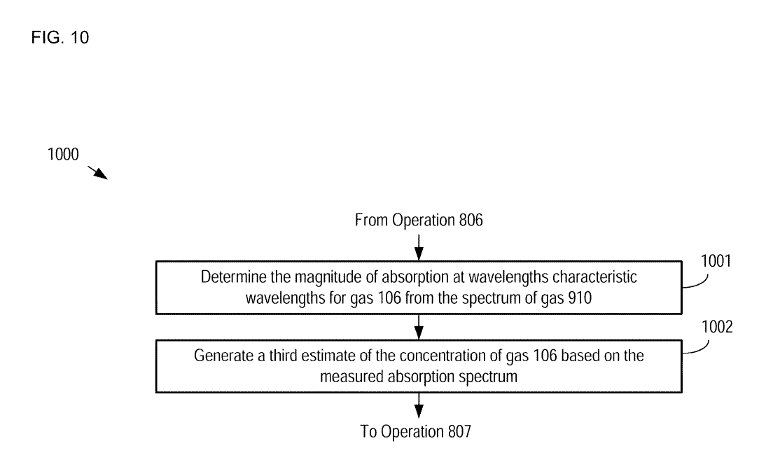

FIG. 10 depicts methods of an operation suitable for detecting a gas via a spectroscopic detection mode. Method 1000 begins with operation 1001, wherein the absorption spectrum of gas 910 is analyzed to determine the magnitude of a plurality of characteristic wavelengths for gas 106.

Upon receiving light signals 716 and 718, optically resonant cavity 908 reflects a portion of each of the light signals as reflected signals 716′ and 718′, where the instantaneous intensity of each reflected light signal is based on the instantaneous cavity length, L, of the optically resonant cavity. As resonator 608 vibrates, cavity length L changes with a periodicity that corresponds to the resonance frequency of the resonator, which gives rise to an intensity modulation of reflected signals 716′ and 718′. Reflected signals 716′ and 718′ are coupled back into optical fiber 604-i via fiber coupler 706.

In order to determine the absorption spectrum of gas 910, reflected signal 718′ is analyzed using a spectrometer included in signal processor 714 for this purpose. One skilled in the art will recognize that as light signals 718 and 718′ pass through gas 910 within optically resonant cavity 908, absorption at a particular set of wavelengths that are characteristic of gas 106 will occur when gas 910 includes gas 106. The absorption of these wavelength signals represents a spectral signature in reflected light signal 718′. It should be noted that spectral analysis of reflected signal 718′ does not require that the gas be interrogated while within an optically resonant cavity and that such interrogation could be performed using, for example, arrangement 900. The multi-reflection that occurs within optically resonant cavity 908, however, magnifies the spectral signal due to absorption, which affords embodiments of the present invention with improved SNR.

At operation 1002, a third estimate of the concentration of gas 106 at location-i is generated based on the measured absorption spectrum of gas 106.

It should be noted that, although operations 1001 and 1002 are described as occurring immediately after operation 806 of method 800, one skilled in the art will recognize that many of the operations of the various methods disclosed herein can be performed in nearly any order.

It is to be understood that the disclosure teaches just one example of the illustrative embodiment and that many variations of the invention can easily be devised by those skilled in the art after reading this disclosure and that the scope of the present invention is to be determined by the following claims.

What is claimed is:

1. An apparatus comprising a first sensor that includes:
a first resonator; and
a first layer disposed on the first resonator, the first layer comprising a plurality of nanoparticles that collectively enable selective chemisorption of a first gas;
wherein a first resonance frequency of the first resonator is based on the mass of the first layer; and
wherein the first sensor is operative for providing a first signal that is indicative of a first concentration of the first gas at a first location.

2. The apparatus of claim 1 further comprising:
a mirror;
a membrane that is movable with respect to the mirror, the first resonator including the membrane, wherein the membrane and first mirror collectively define an optically resonant cavity having a cavity length;
a light source operative for providing a first light signal to the optically resonant cavity;
a spectrometer that is operative for monitoring a plurality of spectral components in a second light signal, wherein the spectrometer is arranged to receive the second light signal from the optically resonant cavity, the second light signal being based on the first light signal and a second gas that is resident in the optically resonant cavity.

3. The apparatus of claim 1 further comprising a second sensor that includes:
a second resonator; and
a second layer disposed on the second resonator, the second layer comprising a plurality of nanoparticles that collectively enable selective chemisorption of a second gas;
wherein a second resonance frequency of the second resonator is based on the mass of the second layer; and
wherein the second sensor is operative for providing a second signal that is indicative of a second concentration of the second gas at a second location.

4. The apparatus of claim 3 wherein the second gas is the same as the first gas.

5. The apparatus of claim 4 further comprising a controller that is operative for (1) receiving the first signal from the first sensor, the first signal being indicative of the concentration of the first gas at a first location, (2) receiving the second signal from the second sensor, the second signal being indicative of the concentration of the first gas at a second location, and (3) generating a spatial map of first-gas concentration based on the first signal and the second signal.

6. The apparatus of claim 1 wherein the first resonator comprises silicon carbide.

7. The apparatus of claim 1 further comprising:
a first electrode; and
a second electrode;
wherein the first and second electrodes are operatively coupled with the first layer such that a first electrical parameter measured between the first and second electrodes is based on the chemisorption of the first gas by the first layer.

8. The apparatus of claim 1 wherein the first sensor further comprises:
a feedback oscillator operative for driving the resonator into resonance; and
a read-out circuit comprising a phase-locked loop.

9. An apparatus comprising:
a first sensor node that includes:
a first gas sensor having a first resonance frequency that is based on the selective chemisorption of a first gas by a first layer;
a first electronic module operative for determining the first resonance frequency; and
a first transceiver for providing a first output signal to a controller, the first output signal being based on the first resonance frequency; and
the controller, wherein the controller is operative for generating an estimate of a first concentration of the first gas at a first location based on the first output signal.

10. The apparatus of claim 9 further comprising a second sensor node that includes:
a second gas sensor having a second resonance frequency that is based on the chemisorption of a second gas by a second layer;
a second electronic module operative for determining the second resonance frequency; and
a second transceiver for providing a second output signal to the controller, the second output signal being based on the second resonance frequency;
wherein the controller is further operative for generating a second estimate of a second concentration of the second gas at a second location based on the second output signal.

11. The apparatus of claim 10 wherein the second gas is the same as the first gas, and wherein the controller is further operative for generating a spatial map of the concentration of the first gas based on the first output signal and the second output signal.

12. The apparatus of claim 9 wherein the first gas sensor includes a first resonator and the first layer, the first layer being disposed on the first resonator, and the first layer comprising a plurality of nanoparticles that collectively enable selective chemisorption of a first gas.

13. The apparatus of claim 9 wherein the first sensor node is dimensioned and arranged to be movable relative to the controller, and wherein the first transceiver is a wireless transceiver.

14. A method comprising:
providing a first layer disposed on a first resonator, the first layer comprising a plurality of nanoparticles that collectively enable selective chemisorption of a first gas;
determining a first resonance frequency of the first resonator, the first resonance frequency being based on the mass of the first layer at a first location; and
estimating a first concentration of the first gas at the first location based on the first resonance frequency.

15. The method of claim 14 further comprising:
providing a second layer disposed on a second resonator, the second layer being selectively chemisorptive for a second gas;
determining a second resonance frequency of the second resonator, the second resonance frequency being based on the mass of the second layer at a second location; and
estimating a second concentration of the second gas at the second location based on the second resonance frequency.

16. The method of claim 15 wherein the second layer is provided such that the second gas is the same as the first gas.

17. The method of claim 16 further comprising generating a spatial map of the concentration based on the first concentration, first location, second concentration, and second location.

18. The method of claim 14 further comprising:
determining a second resonance frequency of the first resonator, the second resonance frequency being based on the mass of the first layer at a second location; and
estimating a second concentration of the first gas at the second location based on the second resonance frequency.

19. The method of claim 14 further comprising:
providing the first resonator such that it defines one surface of an optically resonant cavity having a cavity length that is based on the position of the membrane with respect to a first mirror;
interrogating the optically resonant cavity with a first light signal;
monitoring a plurality of wavelength signals in a second light signal received from the optically resonant cavity, the second light signal being based on the first light signal; and
estimating the concentration of the first gas in the optically resonant cavity based on the plurality of wavelength signals.

20. The method of claim 19 further comprising:
measuring a first electrical parameter of the first layer;
estimating a second concentration of the first gas at the first location based on the first electrical parameter; and
establishing a third concentration of the first gas at the first location based on the first concentration and the second concentration.
